(19) United States
(12) Patent Application Publication
(10) Pub. No.: US 2025/0073692 A1
(43) Pub. Date: Mar. 6, 2025

(54) PROCESS FOR PREPARING LINEAR ALPHA-OLEFIN BY OLIGOMERIZATION OF ETHYLENE

(71) Applicant: NANJING CHEMISTRY NEW ENERGY TECHNOLOGY CO., LTD., Nanjing (CN)

(72) Inventors: Xiwu CHEN, Nanjing (CN); Yi XU, Nanjing (CN); Weituo XIE, Nanjing (CN); Zhen WU, Nanjing (CN); Xiuhong LONG, Nanjing (CN)

(21) Appl. No.: 18/425,918

(22) Filed: Jan. 29, 2024

(30) Foreign Application Priority Data
Aug. 29, 2023 (CN) 202311105152.3

(51) Int. Cl. B01J 37/02; B01J 37/00; C07C 5/333

(52) U.S. Cl. CPC B01J 37/0203; B01J 37/0009; B01J 37/0205; B01J 37/0209; B01J 37/0213; C07C 5/333; C07C 2529/04

(57) ABSTRACT

The present invention relates to a process for preparing a catalyst, wherein the process comprises steps of: 1) providing a molecular sieve, adding an active metal promoter into the molecular sieve, and shaping the molecular sieve into a shaped body, 2) placing the shaped body in a fixed bed reactor, 3) dissolving a transition metal salt in a first valence state and a bidentate ligand into a solvent to prepare a solution, 4) passing the solution and ethylene through the fixed bed reactor charged with the shaped body, loading the transition metal onto the molecular sieve through ion exchange, and at the same time the transition metal in the first valence state at least partially being reduced into a transition metal salt in a second valence state by the active metal promoter, wherein the second valence state is lower than the first valence state, so as to obtain the catalyst.

### CROSS-REFERENCE TO RELATED APPLICATIONS

The application claims the priority date benefit of CN patent application with the application serial number of 202311105152.3, application date of Aug. 29, 2023 with the title of “Process for Preparing Linear alpha-olefin by Oligomerization of Ethylene”. The document of which is incorporated hereto as a whole by reference.

### TECHNICAL FIELD

The present invention relates to a process for preparing linear alpha-olefin by oligomerization of ethylene, and, the present invention particularly relates to a process for preparing linear alpha-olefin by oligomerization of ethylene by using a transition metal ligand catalyst immobilized on a support.

### BACKGROUND OF THE INVENTION

A linear alpha-olefin is a linear olefin containing a double bond at the end of molecule and is an important raw material for the production of a variety of fine chemical products. Linear alpha-olefins are important monomers for preparing low density polyethylene and high density polyethylene; are raw materials for synthesizing high-grade lubricating oils, high-grade surfactants and higher alcohols; and are raw materials for producing products such as various oil-displacing agents, oil additives and the like. In recent years, the demand for linear alpha-olefins is increasing rapidly with the continuous development of the polyolefin industry. Oligomerization of ethylene is the primary process for producing linear alpha-olefins.

Currently, there are many industrialized oligomerization producing technologies for alpha-olefins, including mainly the firstly developed one-step process technology of Chevron Phillips Chemical Company LLC. of USA (CPChem) using triethylaluminum as catalyst, the two-step process technology improved by BP company of UK based on the one-step process, the Shop technology using a nickel complex as catalyst of Shell company of Netherlands, the Idemitsu technology using a zirconium complex as catalyst of Idemitsu Kosan Co., Ltd. of Japan, the Linear-1 technology using a nickel complex coordinated with a special ligand developed jointly by UOP company of USA and Union Carbide company of USA, the α-Sablin technology using zirconium and aluminum bi-components as catalyst of Saudi Basic Industries Corporation of Saudi Arabia, the AlphaSelect technology capable of producing C₄-C₁₀α-olefins of French Institute of Petroleum(IFP), the Versipol technology using a pyridine diimine iron complex as primary catalyst of DuPont company of USA, and the like. At present, the early developed one-step process, two-step method and Shop technology are mainly used in the world, and said three production technologies provide at least 74 percent of the production capacity of alpha-olefin produced by oligomerization process. The Shop technology converts ethylene raw material completely into linear alpha-olefins required by the market via isomerization and olefin metathesis reactions, and has the advantages of low discharge, low pollution, mild operation conditions, safe operation, etc.

In US patents U.S. Pat. Nos. 4,020,121, 4,472,522, and 4,503,279, processes for the preparation of linear alpha-olefins by oligomerization of ethylene were reported by Shell company. The homogeneous catalyst adopted by the processes uses nickel as active component, a diphenylphosphinobenzoate salt as ligand and 1,4-butanediol as solvent. The catalyst uses sodium borohydride as reducing agent to reduce active metal nickel ions. The processes adopt a continuous reaction mode. The solvent is reused by distillation regeneration. The product is washed with regenerated solvent and water to eliminate residual catalyst, so as to inhibit further polymerization of olefins, ensuring product quality and avoiding blocking of the production procedures caused by generation of polymers.

In US patent U.S. Pat. No. 5,523,508, a process for preparing linear alpha-olefins which eliminates wax deposition was reported by UOP company. The process uses transition metal nickel as catalytic active substance, diphenyl(2-naphthyl-1-sulfonic acid)phosphine as ligand and sodium borohydride as activating agent. In this process, a portion of the lighter product components, such as C₁₂-C₁₈ or C₁₂-C₁ᵢ alpha-olefins, which can dissolve the heavier product components sufficiently, is returned to the reaction system, thereby avoiding blocking due to wax deposition.

In US patents U.S. Pat. Nos. 4,686,315 and 4,711,969, a nickel Ylide phosphine catalyst was reported by Chevron company. A certain amount of aluminum alkoxide is added during the oligomerization reaction procedure. The catalyst is featured by a higher reaction activity, but a difficult separation of catalyst from product and a lower product selectivity.

Chinese patent application CN1126107A reported a catalyst for preparing alpha-olefin by oligomerization of ethylene. This application relates to a novel bimetal coordination catalyst for preparing alpha-olefin by ethylene oligomerization. The catalyst consists of a divalent compound of nickel, zinc metal or a monovalent compound thereof, and a water-soluble bidentate organophosphine ligand containing P and O.

Nesterov et al^{[1]} had performed a lot of research work in immobilization of nickel complex catalysts. The work started with loading a tertiary phosphine onto silica gel or alumina. The reaction is as follows:

(E-OH)₂+(EtO)₂Si—C₂H₄—PPh₂→(E-O—)ₙSi(OEt)₃₋ₙ-C₂H₄—PPh₂+nEtOH
n=1, 2; E=Si, Al

The loaded silica gel contains 0.25% P and the loaded alumina contains 0.36% P. The loaded silica gel or alumina reacts with Ni(COD)₂ and O═C(Ph)—CH═PPh₃:

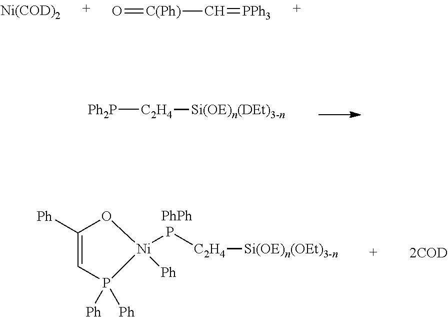

The testing results of the supported solid-phase catalyst show that the activity can be equal to or even higher than the activity of the corresponding homogeneous catalyst under same reaction conditions. However, the selectivity to alpha-olefins is poor, only about 92%.

Marcell Peuckert and Wilhelm Keim^{[2]} prepare a heterogeneous catalyst for preparing linear alpha-olefin by ethylene oligomerization via using a polystyrene resin as solid phase carrier to support active metal nickel. Under the conditions of reaction temperature of 50-75° C. and reaction pressure of 4.0 MPa, a product with linear alpha-olefin content of 99% can be obtained.

The existing industrially operated processes for preparing alpha-olefin by ethylene oligomerization are all homogeneous reaction processes. They have the defect of large heat release during production procedure, affecting product yield and leading to difficult product separation; and require sodium borohydride or aluminum alkoxide as a co-catalyst, reducing the selectivity of the reaction and increasing the production cost.

### REFERENCES

[1] Nesterov G A, Fink G, Heterogenization of a Homogeneous Nickel Chelate Ethylene Oligomerization Catalyst, J Mol Catal, 1991, 66:367-372
[2] Marcell Peuckert and Wilhelm Keim, Journal of Molecular Catalyst, 22 (1984) 289-295

### SUMMARY OF THE INVENTION

This section provides a general summary of the present disclosure, and is not a comprehensive disclosure of its full scope or all of its features.

Further application areas will become apparent from the descriptions provided herein. The descriptions and specific examples in this section are intended for illustration only and are not intended to limit the scope of the present disclosure.

An aim of the present invention is to provide a novel process for preparing catalysts, and the catalysts prepared by the process can be successfully used in the production of linear alpha-olefins via ethylene oligomerization, enabling overcoming of some or even all of the aforementioned drawbacks of the prior art.

According to an aspect the present invention, a process for preparing a catalyst is provided, wherein the process comprises the steps of:
1) providing a molecular sieve, adding an active metal promoter into the molecular sieve, and shaping the molecular sieve into a shaped body,
2) placing the shaped body in a fixed bed reactor,
3) dissolving a transition metal salt in a first valence state and a bidentate ligand into a solvent to prepare a solution,
4) passing the solution and ethylene through the fixed bed reactor charged with the shaped body, loading the transition metal onto the molecular sieve through ion exchange, and at the same time the transition metal in the first valence state at least partially being reduced into a transition metal salt in a second valence state by the active metal promoter, wherein the second valence state is lower than the first valence state, so as to obtain the catalyst.

According to another aspect the present invention, a process for preparing linear alpha-olefin by oligomerization of ethylene using the above catalyst.

It has been surprisingly found that the process according to the present invention can prepare heterogeneous catalysts in a simple manner, which catalysts can be advantageously used in industrially operated processes for preparation of alpha-olefins by ethylene oligomerization and can lead to an advantageous heat diffusion and facilitate an efficient separation of the catalyst from the product. The preparation process of the catalyst and the subsequent oligomerization process do not need to use expensive and in some cases dangerous co-catalysts sodium borohydride or aluminum alkoxide, and accordingly omit the troublesome subsequent separation post-treatment of the co-catalyst. Furthermore, the heterogeneous catalysts show equal or even higher catalytic activity and selectivity for linear alpha-olefins compared with homogeneous catalysts.

### BRIEF DESCRIPTION OF DRAWINGS

FIG. 1 shows a gas chromatogram chart for a typical product according to the present invention.

### DETAILED DESCRIPTION OF THE INVENTION

In the context of this disclosure, the disclosure of a range includes the disclosure of all values and further subdivided ranges within the entire range, including the endpoints given to these ranges and sub-ranges.

The present invention relates to the following aspects:
1. A process for preparing a catalyst, wherein the process comprises the steps of:
1) providing a molecular sieve, adding an active metal promoter into the molecular sieve, and shaping the molecular sieve into a shaped body,
2) placing the shaped body in a fixed bed reactor,
3) dissolving a transition metal salt in a first valence state and a bidentate ligand into a solvent to prepare a solution,
4) passing the solution and ethylene through the fixed bed reactor charged with the shaped body, loading the transition metal onto the molecular sieve through ion exchange, and at the same time the transition metal in the first valence state at least partially being reduced into a transition metal salt in a second valence state by the active metal promoter, wherein the second valence state is lower than the first valence state, so as to obtain the catalyst.
2. The process according to aspect 1, wherein the fixed bed reactor is preferably of tubular type.
3. The process according to any one of the preceding aspects, wherein the fixed bed reactor is preferably jacket heat exchanged.
4. The process according to any one of the preceding aspects, wherein the transition metal salt is selected from the group consisting of nickel chloride, nickel bromide, nickel iodide, nickel nitrate, nickel carbonate, nickel chlorate and the like, preferably nickel chloride.
5. The process according to any one of the preceding aspects, wherein the bidentate ligand is an organophosphine ligand selected from the group consisting of diphenylphosphinocarboxylic acid, sodium diphenylphosphinocarboxylate, diphenylphosphinoacetic acid, sodium diphenylphosphinoacetate, diphenylphosphinobenzoic acid and sodium diphenylphosphinobenzoate, preferably sodium diphenylphosphinobenzoate.
6. The process according to any one of the preceding aspects, wherein the organic solvent is selected from the group consisting of cyclopentane, cyclohexane, isooctane, decane, benzene, toluene, ethylbenzene, methanol, ethanol, n-propanol, n-butanol, octanol, dodecanol, ethylene glycol, propylene glycol, butylene glycol, pentylene glycol, hexylene glycol, preferably 1,4-butanediol and 2,5-hexanediol, more preferably 1,4-butanediol.
7. The process according to any one of the preceding aspects, wherein the molar ratio of metallic nickel to ligand is 0.5-10, preferably 1-3, and most preferably 2.
8. The process according to any one of the preceding aspects, wherein the temperature at which the ion exchange is carried out is from 10 to 50° C., preferably from 20 to 40° C. and most preferably 30° C.
9. The process according to any one of the preceding aspects, wherein the partial pressure of ethylene during ion exchange is from 0.5 to 5 MPa, preferably from 2 to 4 MPa, and most preferably 3 MPa.
10. The process according to any one of the preceding aspects, wherein the ion exchange is carried out for a period of 1 to 20 hours, preferably 5 to 10 hours, and most preferably 8 hours.
11. The process according to any one of the preceding aspects, wherein the molecular sieve is selected from the group consisting of X, Y, ZSM-5, L, MCM-22 and MCM-36 type molecular sieves, preferably L type molecular sieve.
12. The process according to any one of the preceding aspects, wherein the active metal promoter is selected from the group consisting of metal Al, Zn, Fe, Cd, and Co, preferably Zn.
13. The process according to aspect 12, wherein the content of the active metal promoter is 5 to 70 wt %, preferably 10 to 40 wt %, and most preferably 22 wt %, relative to the total weight of the solid catalyst prepared.
14. The process according to any one of the preceding aspects, wherein the shaping method of the solid catalyst is compression shaping method, extrusion shaping method or rotational shaping method, preferably compression shaping method.
15. The process according to any one of the preceding aspects, wherein the loading amount of nickel is from 0.1 to 5 wt %, preferably from 1 to 3 wt %, relative to the total weight of the solid catalyst prepared.
16. According to an aspect of the present invention, a catalyst is provided, which catalyst is prepared by the process according to any one of the preceding aspects.
17. According to an aspect of the present invention, use of the catalyst according to the present invention for oligomerization of ethylene is provided.
18. A process for preparing linear alpha-olefin by oligomerization of ethylene, which process comprises passing ethylene continuously through a fixed bed reactor charged with a catalyst, to oligomerize to prepare the linear alpha-olefin, characterized in that the catalyst is prepared in the following way:
1) providing a molecular sieve, adding an active metal promoter into the molecular sieve, and shaping the molecular sieve into a shaped body,
2) placing the shaped body in a fixed bed reactor,
3) dissolving a transition metal salt in a first valence state and a bidentate ligand into a solvent to prepare a solution,
4) passing the solution and ethylene through the fixed bed reactor charged with the shaped body, loading the transition metal onto the molecular sieve, and at the same time the transition metal in the first valence state at least partially being reduced into a transition metal salt in a second valence state by the active metal promoter, wherein the second valence state is lower than the first valence state.
19. The process according to aspect 18, wherein the temperature for oligomerization is 50 to 120° C., preferably 70 to 100° C., and most preferably 90° C.
20. The process according to aspect 18 or 19, wherein the pressure for oligomerization is 6-12 MPa, preferably 8-11 MPa, and most preferably 10 MPa.

### I. Preparation of Catalysts

According to an aspect of the present invention, a process for preparing a catalyst is provided, wherein the process comprises the steps of:
1) providing a molecular sieve, adding an active metal promoter into the molecular sieve, and shaping the molecular sieve into a shaped body,
2) placing the shaped body in a fixed bed reactor,
3) dissolving a transition metal salt in a first valence state and a bidentate ligand into a solvent to prepare a solution,
4) passing the solution and ethylene through the fixed bed reactor charged with the shaped body, loading the transition metal onto the molecular sieve through ion exchange, and at the same time the transition metal in the first valence state being reduced into a transition metal in a second valence state by the active metal promoter, wherein the second valence state is lower than the first valence state, so as to obtain the catalyst.

In the first step of the process for preparing catalyst of the invention, an active metal promoter is added into the molecular sieve, and the molecular sieve is placed in a fixed bed reactor after shaping. The fixed bed reactor can be of a type of tubular, horizontal, vertical and loop, and is preferably tubular type. The heat exchange mode is preferably a jacket heat exchange mode. The heat exchange medium is not particularly limited and can be a common heat exchange medium in the art, such as water.

In the context of the present invention, the molecular sieve refers to hydrated aluminosilicates. The molecular sieves used in the present invention include X-type molecular sieve, Y-type molecular sieve, ZSM-5 type molecular sieve, L-type molecular sieve, MCM-22 type molecular sieve, MCM-36 type molecular sieve and the like, and the L-type molecular sieve is preferable.

In the context of the present invention, the active metal promoter includes all metals that can reduce the transition metal from the first valence state to the second valence state under the conditions for preparing the catalyst. The active metal promoter used in the present invention comprises Al, Zn, Fe, Cd and Co in metal form, and preferably Zn in metal form. Zn is preferably in the form of powder to increase the contact area so as to facilitate the reduction reaction. The content of the active metal promoter is 5 to 70 wt %, preferably 10 to 40 wt %, and most preferably 22 wt %, relative to the total weight of the solid catalyst prepared.

The shaping method for the solid catalyst adopted in the present invention is compression shaping method, extrusion shaping method or rotational shaping method. Compression shaping method is preferred.

During the shaping procedure, a lubricant can optionally be added to the catalyst to facilitate demoulding, and examples of the lubricant include graphite, sesbania powder, and talc powder. Graphite is preferred.

A transition metal salt and an organic bidentate ligand in a certain ratio are dissolved into an organic solvent to prepare a solution. The solution is passed through a fixed bed reactor charged with the shaped body under conditions of a certain temperature, pressure and presence of ethylene, the transition metal (primary catalyst) is loaded onto the molecular sieve via ion exchange. And at the same time, the transition metal (primary catalyst) in the first valence state is at least partially reduced to a second valence state by the active metal promoter. Wherein the second valence state is preferably lower than the first valence state. The first valence state is preferably positive bivalent. The second valence state can be monovalent or zero valent, preferably zero valent. The loading amount of the transition metal salt (primary catalyst) on the molecular sieve is controlled by controlling the exchange time.

Transition metal nickel can form a planar quadrilateral structure with a bidentate ligand due to its unique valence electronic structure, and is one of the most important catalyst active substances for synthesizing linear alpha-olefin by ethylene oligomerization. The nickel salts used in the process of present invention for preparing the catalyst mainly comprise divalent metal salts of nickel, such as nickel chloride, nickel bromide, nickel iodide, nickel nitrate, nickel carbonate, nickel chlorate and the like, and nickel chloride is preferred.

The bidentate ligand mainly refers to P—O type organophosphines, mainly including diphenylphosphinocarboxylic acid, diphenylphosphinoacetic acid, diphenylphosphinobenzoic acid, sodium diphenylphosphinocarboxylate, sodium diphenylphosphinoacetate, sodium diphenylphosphinobenzoate, potassium diphenylphosphinocarboxylate, potassium diphenylphosphinoacetate, potassium diphenylphosphinobenzoate, lithium diphenylphosphinocarboxylate, lithium diphenylphosphinoacetate, lithium diphenylphosphinobenzoate, ammonium diphenylphosphinocarboxylate, ammonium diphenylphosphinoacetate, ammonium diphenylphosphinobenzoate, and the like, preferably sodium diphenylphosphinobenzoate.

The organic solvent can be a nonpolar solvent such as cyclopentane, cyclohexane, isooctane, decane, benzene, toluene, ethylbenzene, and the like; or a polar solvent such as methanol, ethanol, n-propanol, n-butanol, octanol, dodecanol, ethylene glycol, propylene glycol, butylene glycol, pentylene glycol, hexylene glycol, and the like, preferably 1,4-butanediol, 2,5-hexanediol, and the like, more preferably 1,4-butanediol.

The molar ratio of the nickel to the ligand added in the step 3) is 0.5-10, preferably 1-3, and most preferably 2.

Molecular sieves have larger specific surface areas, regular channel structures and adjustable channel sizes; have appropriate surface acidities and the surface acidities can be conveniently adjusted according to the needs of reaction; and have extremely strong screening effect on hydrocarbon molecules. Therefore, molecular sieves are carrier materials for ethylene oligomerization reaction with a wide application prospect. Although not constituting a limitation to the inventive concept of present application, it is believed that ethylene can react both within channels of and on the outer surfaces of the molecular sieves.

The temperature for the ion exchange of nickel is 10-50° C., preferably 20-40° C., and most preferably 30° C.

The partial pressure of ethylene during exchange is from 0.5 to 5 MPa, preferably from 2 to 4 MPa, and most preferably 3 MPa.

The exchange time is 1 to 20 hours, preferably 5 to 10 hours, and most preferably 8 hours.

In the catalyst obtained by the process of the present invention, the loading amount of nickel is from 0.1 to 5 wt %, preferably from 0.15 to 3 wt %, relative to the total weight of the solid catalyst prepared.

The catalyst according to the present invention is generated directly in the reactor in an in-situ generation manner, thereby avoiding loss, deterioration and pollution of the catalyst during storage and transportation.

### II. Oligomerization of Ethylene

According to an aspect the present invention, a process for preparing a linear alpha-olefin by oligomerization of ethylene using the above catalyst is provided. The disclosure presented above with respect to the catalyst applies correspondingly to the ethylene oligomerization process according to the present invention and thus will not be repeated in this section.

After the completion of preparation of catalyst, the raw material ethylene for the reaction is passed continuously through a fixed bed reactor at a certain space velocity, and the oligomerization of ethylene is carried out under certain temperature and pressure conditions so as to prepare linear alpha-olefin.

The temperature for oligomerization is 50 to 120° C., preferably 70 to 100° C., and most preferably 90° C.

The reaction pressure of ethylene is 6-12 MPa, preferably 8-11 MPa, and most preferably 10 MPa.

The process according to the present invention typically produces a variety of C₄ to C₄₈ linear alpha-olefins, wherein the product distribution conforms to the Schulz-Flory model and the geometric growth factor K value is a characterization of the relative proportion of product olefins.

$$K = \frac{molar amount of {C}_{n + 2} olefin}{molar amount of {C}_{n} olefin} ( n = 4 , 6 , 8 , … )$$

The K-value of the ethylene oligomers obtained by the process according to the present invention is in the range of from 0.40 to 0.90, preferably in the range of from 0.60 to 0.80.

### EXAMPLES

The following examples are further exemplary illustration of the present invention and are not intended to limit the scope of the present invention. The following exemplary examples are provided so that this disclosure will be thorough, and will fully convey the scope to those skilled in the art. Numerous specific details are set forth such as examples of specific compositions, components, devices, and methods, to provide a thorough understanding of embodiments of the present disclosure. It will be apparent to those skilled in the art that, without the need of specific details, that exemplary examples may be embodied in many different forms and that neither should be construed to limit the scope of the disclosure. In some exemplary examples, well-known processes, well-known device structures, and well-known technologies are not described in detail.

### Analysis of Product Composition

The analysis of each product was performed by gas chromatography. Exemplary analytical conditions for gas chromatography are: 30 m×0.32 mm×0.1 μm SE-30 capillary chromatographic column, sample injector temperature 300° C., detector temperature 300° C., split ratio 50.00, and column pressure 1.004 psi. A programmed heating was used for the column temperature: 30-200° C., ramp rate 10° C./min, 200-340° C., ramp rate 3° C./min.

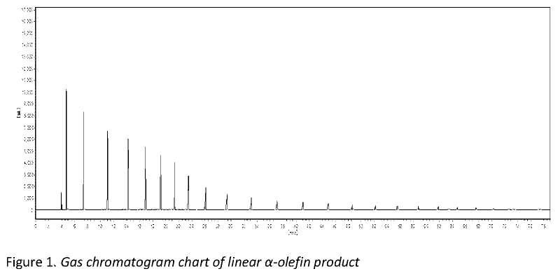

A typical gas chromatogram for the product is shown in FIG. 1.

### Determination of Nickel Ion Content

The content analysis of nickel ion was determined by Inductively Coupled Plasma Spectrometry (ICP). The apparatus used was: ICP-OES model Avio 200. A solid state RF generator was used with a power of 750-1500 W. The monochromator adopts an echelle grating and prism two-dimensional dispersion system with a wavelength range of 170-750 nm and a focal length of 200-650 nm. The detector is a solid state detector with a resolution of <0.003 nm.

### Example 1

### Preparation of Solid Catalysts with Different Types of Molecular Sieves as Carriers and Reaction Results

100 g of X, Y, ZSM-5, L, MCM-22 and MCM-36 type molecular sieves were respectively weighed as different type carriers, and 30 g of zinc powder and 5 g of graphite were respectively added to each carrier. The mixtures were pressed into Φ5×5 mm tablets by a tablet press, and the tablets were marked as samples A1, A2, A3, A4, A5 and A6 respectively.

8.5 g of NiCl₂·6H₂O salt and 5.5 g of sodium diphenylphosphinobenzoate were weighed and dissolved into 1000 g of 1,4-butanediol to form the exchange solution W.

100 g of sample A1 was placed in a jacket heat-exchanged tubular type fixed bed reactor, and ethylene gas was introduced while maintaining the ethylene pressure at 3.0 MPa. The exchange solution W was injected into the reactor by a metering pump, and the flow rate of the metering pump was controlled at 1000 g/h. The solution was recycled. The temperature was maintained at 30° C. and the exchanging was conducted continuously for 8 hours. After the exchange was completed, the ethylene pressure was raised to 9.0 MPa, the reaction temperature was raised to 90° C. and the ethylene feed rate was controlled at 60 g/h.

The stream discharged from the fixed bed reactor was firstly subjected to gas-liquid separation, and the unreacted ethylene gas was returned back to the reaction system for recycling. The alpha-olefin product in the liquid phase was not miscible with the solvent and stratified naturally. The upper layer product was collected for analysis. The lower layer solution was returned back to the reaction system for recycling.

The reaction was conducted continuously for 10 hours and samples were taken for analysis such that reaction results for sample A1 were obtained.

The above procedures were repeated to obtain the reaction results for samples A2, A3, A4, A5 and A6, as shown in Table 1.

The experimental results show that the solid phase catalyst prepared by the L-type molecular sieve provides better reaction activity and selectivity.

### Example 2

### Preparation of Solid Catalysts with Different Contents of Active Metal Promoter and Reaction Results

100 g of L-type molecular sieve was weighed, 5 g of graphite was added, and 5 g, 10 g, 20 g, 30 g, 40 g and 50 g of zinc powder were respectively added. The mixtures were pressed into Φ5×5 mm tablets by a tablet press, and the tablets were marked as samples B1, B2, B3, A4, B4 and B5 respectively.

The loading of the active component nickel and the reaction procedures are the same as those of the Example 1.

The reaction results are shown in Table 2.

The reactivity of catalyst firstly increases and finally reaches a plateau, and the selectivity of the reaction decreases, with the increase of the content of active metal promoter zinc.

### Example 3

### Effect of Different Nickel Loading Amounts on Reaction Results

The loading amount of the active component nickel on the solid catalyst was controlled by the time of ion exchange. A4 sample was used as solid phase catalyst, and the content of nickel ions in the circulating liquid after different exchange times was determined, thereby the nickel loading amount on the solid catalyst was calculated. The loading procedure of the nickel and the reaction procedure are the same as those of the Example 1, except the loading time.

The loading amounts of nickel in the solid catalysts and the reaction results are shown in Table 3.

As the exchange time increases, the content of nickel on the solid phase catalyst increases and the reaction reactivity increases. And the reaction reactivity descends after the highest reaction reactivity is reached. This is because the oligomerization reaction of ethylene is carried out under the combined action of active metal nickel and acid sites for a supported catalyst. At the beginning, as the nickel loading amount increases, the number of metal active sites increases and accordingly the activity of polymerization reaction increases. When the amount of active metal nickel is increased to a certain amount, some metal nickel ions will cover a part of the surface acid sites of the molecular sieve, thereby inhibiting the activity of the polymerization reaction.

### Example 4

### Effect of Different Reaction Temperatures on the Oligomerization Reaction

A4 sample was selected as solid phase catalyst for evaluating the reaction, and the method of active metal ion exchange and the reaction procedures are the same as those in Example 1. The effect of different reaction temperatures on the activity and selectivity was evaluated. The reaction results are shown in Table 4.

### Example 5

### Effect of Different Reaction Pressures on the Oligomerization Reaction

A4 sample was selected as solid phase catalyst for evaluating the reaction, and the method of active metal ion exchange and the reaction procedures are the same as those in Example 1. The effect of different pressures on the activity and selectivity was evaluated. The reaction results are shown in Table 5.

The ethylene oligomerization reaction is a reaction with reduced number of molecules, and accordingly the oligomerization reaction is facilitated by increasing the reaction pressure. However, too high a reaction pressure increases the requirements for the strength of catalyst and the equipment for reaction, and accordingly increases the production cost.

TABLE 1

| | productivity (mg | selectivity | product |
|---|---|---|---|
| | product/g solid | (linear | distribution |
| samples | catalyst · h) | α-olefin %) | K value |
| A1 | 356.6 | 96.2 | 0.79 |
| A2 | 372.5 | 97.3 | 0.77 |
| A3 | 365.3 | 97.5 | 0.73 |
| A4 | 405.1 | 99.1 | 0.78 |
| A5 | 413.6 | 96.4 | 0.72 |
| A6 | 428.7 | 96.0 | 0.73 |

TABLE 2

| | productivity (mg | selectivity | product |
|---|---|---|---|
| | product/g solid | (linear | distribution |
| samples | catalyst · h) | α-olefin %) | K value |
| B1 | 246.3 | 99.2 | 0.77 |
| B2 | 301.3 | 99.1 | 0.74 |
| B3 | 383.5 | 99.0 | 0.78 |
| A4 | 405.1 | 99.1 | 0.78 |
| B4 | 410.4 | 98.4 | 0.73 |
| B5 | 407.6 | 98.0 | 0.72 |

TABLE 3

| | Loading | | | |
|---|---|---|---|---|
| exchange | amount | productivity (mg | selectivity | product |
| time | of nickel | product/g solid | (linear | distribution |
| (hours) | (wt. %) | catalyst · h) | α-olefin %) | K value |
| 2 | 0.33 | 86.2 | 99.3 | 0.77 |
| 4 | 0.58 | 135.5 | 99.2 | 0.74 |
| 6 | 0.84 | 311.2 | 99.1 | 0.73 |
| 8 | 1.20 | 405.1 | 99.1 | 0.78 |
| 10 | 1.26 | 397.8 | 99.2 | 0.75 |
| 12 | 1.38 | 355.5 | 99.1 | 0.79 |

TABLE 4

| reaction | productivity (mg | selectivity | product |
|---|---|---|---|
| temperature | product/g solid | (linear | distribution |
| (° C.) | catalyst · h) | α-olefin %) | K value |
| 60 | 83.6 | 99.5 | 0.75 |
| 70 | 158.5 | 99.4 | 0.77 |
| 80 | 387.3 | 99.2 | 0.79 |
| 90 | 405.1 | 99.1 | 0.78 |
| 100 | 399.1 | 98.3 | 0.77 |
| 110 | 386.8 | 97.1 | 0.76 |

TABLE 5

| reaction | productivity (mg | selectivity | product |
|---|---|---|---|
| pressure | product/g solid | (linear | distribution |
| (MPa) | catalyst · h) | α-olefin %) | K value |
| 5.0 | 135.6 | 99.2 | 0.73 |
| 6.0 | 244.7 | 99.2 | 0.73 |
| 7.0 | 368.9 | 99.3 | 0.74 |
| 8.0 | 387.8 | 99.1 | 0.77 |
| 9.0 | 405.1 | 99.1 | 0.78 |
| 10.0 | 411.5 | 99.2 | 0.80 |

The foregoing description of the embodiments has been provided for purposes of illustration and description. It is not intended to be exhaustive or to limit the present disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A process for preparing a catalyst, wherein the process comprises the steps of:
1) providing a molecular sieve, adding an active metal promoter into the molecular sieve, and shaping the molecular sieve into a shaped body,
2) placing the shaped body in a fixed bed reactor,
3) dissolving a transition metal salt in a first valence state and a bidentate ligand into a solvent to prepare a solution,
4) passing the solution and ethylene through the fixed bed reactor charged with the shaped body, loading the transition metal onto the molecular sieve through ion exchange, and at the same time the transition metal in the first valence state at least partially being reduced into a transition metal salt in a second valence state by the active metal promoter, wherein the second valence state is lower than the first valence state, so as to obtain the catalyst.

2. The process according to claim 1, wherein the fixed bed reactor is of tubular type.

3. The process according to claim 1, wherein the fixed bed reactor is jacket heat exchanged.

4. The process according to claim 1, wherein the transition metal salt is selected from the group consisting of nickel chloride, nickel bromide, nickel iodide, nickel nitrate, nickel carbonate, and nickel chlorate.

5. The process according to claim 1, wherein the bidentate ligand is an organophosphine ligand selected from the group consisting of diphenylphosphinocarboxylic acid, sodium diphenylphosphinocarboxylate, diphenylphosphinoacetic acid, sodium diphenylphosphinoacetate, diphenylphosphinobenzoic acid and sodium diphenylphosphinobenzoate.

6. The process according to claim 1, wherein the organic solvent is selected from the group consisting of cyclopentane, cyclohexane, isooctane, decane, benzene, toluene, ethylbenzene, methanol, ethanol, n-propanol, n-butanol, octanol, dodecanol, ethylene glycol, propylene glycol, butylene glycol, pentylene glycol, hexylene glycol.

7. The process according to claim 1, wherein the molar ratio of metallic nickel to ligand is 0.5 to 10.

8. The process according to claim 1, wherein the temperature at which the ion exchange is carried out is from 10° C. to 50° C.

9. The process according to claim 1, wherein the partial pressure of ethylene during the ion exchange is from 0.5 MPa to 5 MPa.

10. The process according to claim 1, wherein the ion exchange is carried out for a period of 1 hour to 20 hours.

11. The process according to claim 1, wherein the molecular sieve is selected from the group consisting of X, Y, ZSM-5, L, MCM-22 and MCM-36 type molecular sieves.

12. The process according to claim 1, wherein the active metal promoter is selected from the group consisting of metal Al, Zn, Fe, Cd, and Co.

13. The process according to claim 12, wherein the content of the active metal promoter is 5 wt % to 70 wt %.

14. The process according to claim 1, wherein the shaping method of the solid catalyst is a compression shaping method, an extrusion shaping method or a rotational shaping method.

15. A process for preparing linear alpha-olefin by oligomerization of ethylene, which process comprises passing ethylene continuously through a fixed bed reactor charged with a catalyst for oligomerizing to prepare the linear alpha-olefin,
characterized in that the catalyst is prepared in the following way:
1) providing a molecular sieve, adding an active metal promoter into the molecular sieve, and shaping the molecular sieve into a shaped body,
2) placing the shaped body in a fixed bed reactor,
3) dissolving a transition metal salt in a first valence state and a bidentate ligand into a solvent to prepare a solution,
4) passing the solution and ethylene through the fixed bed reactor charged with the shaped body, loading the transition metal onto the molecular sieve through ion exchange, and at the same time the transition metal in the first valence state is at least partially being reduced into a transition metal salt in a second valence state by the active metal promoter, wherein the second valence state is lower than the first valence state.

16. The process according to claim 15, wherein the temperature for oligomerization is 50° C. to 120° C.

17. The process according to claim 15, wherein the pressure for oligomerization is 6 MPa to 12 MPa.
